(19) United States
(12) Patent Application Publication
(10) Pub. No.: US 2007/0295115 A1
(43) Pub. Date: Dec. 27, 2007

(54) ASSEMBLY AND METHOD FOR ACCELERATED WEATHERING WITH AN AUTOMATED PROGRAMMABLE CYCLE

(75) Inventors: Henry K. Hardcastle, Phoenix, AZ (US); Duncan I. Maciver, Phoenix, AZ (US); Joseph Alva Farley, Black Canyon City, AZ (US)

Correspondence Address: VEDDER PRICE KAUFMAN & KAMMHOLZ, 222 N. LASALLE STREET, CHICAGO, IL, 60601, US

(73) Assignee: Atlas Material Testing Technology LLC, Chicago, IL (US)

(21) Appl. No.: 11/425,993

(22) Filed: Jun. 22, 2006

(51) Int. Cl. G01N 33/00

(52) U.S. Cl. 73/866

(57) ABSTRACT

An apparatus and method for outdoor accelerated weathering of a test specimen disposed on an accelerated weathering test apparatus. A controller actuates a tracking servo-motor so the test specimen may be periodically exposed to concentrated sunlight and removed therefrom during a test period. A radiant exposure logging assembly logs a light dose measured by a radiant exposure measuring device that impinges on the test specimen over the test period.

### BACKGROUND

The present disclosure generally relates to accelerated weathering device and methods of operation thereof, and more particularly, to an improved outdoor accelerated weathering apparatus and method including an automated programmable cycle for exposure to concentrated sunlight and moisture.

Conventional methods for applying a water spray to test specimens are generally set forth in the standard ASTM G90, which is incorporated herein by this reference. The structure associated with such method includes an independently controlled water spray system which is connected to a device for exposing test specimens to concentrated sunlight as shown in FIG. 1 and described in more detail below.

A conventional apparatus includes a water supply hose attached to a pipe with spray nozzles that are aimed at the test specimens for wetting while being exposed to concentrated sunlight. A solenoid valve controls the flow of water to the spray nozzles. A simple timer is connected to the solenoid valve and programmed with different spray frequencies and durations.

In accordance with the standard ASTM G90, three conventional primary water spray schedules have been developed and used: (1) concentrated sunlight exposures with no water spray; (2) day time spray cycles where water is applied to specimens at the same time the specimens are exposed to concentrated sunlight as shown in FIGS. 3A-C; and (3) night time spray cycles where water is applied to specimens after concentrated sunlight devices are disabled because sunlight is unavailable as shown in FIGS. 4A-C.

Referring to FIG. 3A, an operative portion 100 of an accelerated weathering test device is disposed such that the mirrors 34, 36 are positioned to reflect solar radiation 39 to directly impinge upon the test specimen 46. Referring to FIG. 3B, a water spray 102 is emitted from the water spraying nozzle assembly 51 while the test specimens 46 are exposed to concentrated sunlight 39. Referring to FIG. 3C, the water spray assembly 51 has been deactivated and the test specimens 46 continue to be exposed to concentrated sunlight 39.

Referring to FIG. 4A, the operative portion 100 of the accelerated weathering test device is disposed such that the mirrors 34, 36 are positioned such that concentrated sunlight 39 directly impinges on the test specimen 46. Referring to FIG. 4B, when sunlight is no longer available, the operative portion 100 is disposed in a vertically aligned position such that the water spray assembly 51 may be activated to expose the test specimens 46 to a water spray 102. Referring to FIG. 4C, when solar exposure is again available, the operative portion 100 is returned to a position as described with respect to FIG. 4A and the test specimens 46 are again exposed to concentrated sunlight 39.

There are several disadvantages of these prior art apparatus and methods for exposing test specimens to moisture. One such disadvantage is that the water spray system is not integrated into the operation of the tracking or exposure accounting systems for the concentrated sunlight devices. The conventional structure is to simply attach an independent water spray system to the concentrated sunlight exposure device.

Another disadvantage of conventional exposure methods is that the duration and frequency of periods when the test specimens are exposed to concentrated sunlight and the water spray are not readily variable. The only options available to expose test specimens to moisture under conventional methods were to spray the test specimens under full concentrated sunlight exposure or wait until a regular period of darkness when the machines are regularly turned off, i.e., typically night time, when no sun was available at all.

Those of skill in the art recognize the many disadvantages of wetting the test specimens while simultaneously exposing them to concentrated sunlight and hence, the night time water spray cycle was incorporated into the old method. However, as will be recognized by one of skill in the art, there is a distinct disadvantage associated with the night time water spray cycle. Namely, after the test specimen absorbs the desired amount of moisture, the test specimen may not be subsequently exposed to concentrated sunlight until the night time period has ceased accordingly. Only one cycle per day may be achieved.

One disadvantage associated with a method of wetting specimens while simultaneously exposing them to concentrated sunlight is that spraying cool water onto relatively hot test specimen surfaces result in a thermo-shock which may unnaturally effect the degrading surfaces under test conditions. Concentrated sunlight devices focus considerable energy on the test specimens and raise surface temperatures thereof well above ambient temperatures to the point where a blower is used to circulate air across the surface of the test specimen to keep the test specimens within their temperature operating limits when exposed to concentrated sunlight. Spraying water onto these hot surfaces may result in unnatural thermo-radiance and may result in mechanical stresses which may degrade exposure test specimens in an unnatural manner.

Another disadvantage of applying water spray to test specimens while simultaneously exposing them to concentrated sunlight is that the blower that circulates cooling air usually remains on in the conventional methods. Spraying water into a blown air stream (especially in desert environments where the substantial majority of concentrated sunlight devices are typically installed) results in additional evaporative cooling of exposure test specimens further increasing the unnatural thermo-mechanical stresses experienced by the test specimen.

Another disadvantage of applying water spray to test specimens while simultaneously exposing them to concentrated sunlight, is that this conventional method does not simulate the natural occurrence of moisture exposure in end use conditions. First, precipitation cycles usually occur under cloudy conditions in end use. Therefore, materials have a chance to cool near ambient air temperature before being exposed to moisture by precipitation and natural end use situations. Second, in many end use environments, the majority of moisture exposure time or time of wetness is not due to precipitation, but rather due to night-time condensation end use patterns, specimens are not exposed to sunlight until the end of the night condensation when the sun comes up and specimens dry due to solar heating.

Another disadvantage of wetting specimens while simultaneously exposing them to concentrated sunlight is the lensing effect, where hemispherical-shaped water droplets on the material surface may further concentrate the concentrated sunlight onto discreet areas on the surface of the test specimen causing non-uniform areas of material degradation across the test specimen surface.

Another disadvantage of applying wetting spray while the concentrated sunlight device is turned off at night, is that one cannot produce the same proportion of radiant exposure (light dose) to wet time needed to simulate this proportion observed in the end use environment. For example, it is known in South Florida that a material being exposed will receive approximately 1 MJ/m̂2 ultraviolet radiant exposure each summer day followed by a time of wetness each evening. Accordingly, the natural exposure ratio is approximately 1 MJ/m̂2 ultraviolet to 1 wet cycle in a twenty-four period. The conventional exposure and wetting methods will not allow this proportion to be simulated on a concentrated sunlight device with night time wetting. For example, it is known that on an existing concentrated sunlight device that a material being exposed to concentrated sunlight may receive approximately 5 MJ/m̂2 ultraviolet radiant exposure each summer day and using the night time wetting, must wait until night for the spray. The ratio of 5 MJ/m̂2 ultraviolet radiant exposure to 1 wet cycle of the conventional method is very different than the exposure/wet cycle ratio observed in the materials in end use environment. Therefore, the degradation rates observed with the conventional methods may not closely simulate nor correlate with the end use exposure behavior.

Another disadvantage of the conventional night time wetting method is that such method does not efficiently time wet spray or take into account the moisture absorption characteristics of the material. For instance, some materials absorb water more quickly than other materials when exposed to the water spray. If the test specimen has a moisture absorption rate that allows its to absorb a critical amount of water in thirty minutes (i.e., reach eighty percent of saturation level) this material may reach this critical amount each night during a natural exposure (such as in South Florida where wet time regularly exceeds five hours each night). However, using the conventional method with a fifteen minute spray, the test material may never actually reach the critical point of absorbed moisture.

Another disadvantage of the conventional methods is that the servo-motors are only controlled to either to track the sun or to turn on or turn off the concentrated sunlight devices. The servo-motors are not controlled to move specimens out of the concentrated natural sunlight to allow for a period of unexposed concentrated sunlight in order to spray the test specimen with a fluid. Conventional methods did not permit such unexposed periods to be integrated into the control of the device as a controlled exposure variable.

Another disadvantage of the conventional methods is that the accounting of radiant exposure (light dose) is not integrated into control of the concentrated sunlight device. Conventionally, the radiant exposure is measured using an independent photosensor exposed to direct sunlight and an independent data logging system. The standard ASTM G90 describes the independent radiant exposure tracing system. Such conventional methods fail to provide an integrated system for cycling test specimens between exposed and unexposed periods with respect to concentrated sunlight on concentrated sunlight devices and recording such exposed and unexposed periods with respect to radiant exposure.

Therefore, there exists a need in the art for an apparatus and method for an accelerated weathering device with an automated programmable cycle for exposure to concentrated sunlight and moisture that overcomes the disadvantages of conventional apparatus and methods and incorporates the advantageous features disclosed herein.

### SUMMARY

In accordance with one principal aspect of the present disclosure, an accelerated weathering assembly comprises an outdoor accelerated weathering test apparatus including a test specimen and a tracking servo-motor connected thereto. A controller is connected to the tracking servo-motor so that the test specimen may be exposed to concentrated sunlight for an exposure portion of a test period and removed from exposure to the concentrated sunlight for an unexposed portion of the test period. Such actions may be repeated at a desired frequency and duration for the remainder of the test period. A radiant exposure logging assembly comprises a radiant exposure measuring device connected to a recording device which logs a light dose measured by the radiant exposure measuring device that impinges on the test specimen over the test period.

In accordance with another principal aspect of the present disclosure, a method for accelerated weathering of a test specimen on an outdoor accelerated weathering test apparatus for a test period is disclosed. A radiant exposure measuring device is disposed adjacent the test specimen. A recording device is connected to the radiant exposure measuring device. A controller is connected to a tracking servo-motor so that the test specimen may be exposed to concentrated sunlight for an exposure portion of the test period and removed from exposure to the concentrated sunlight for an unexposed portion of the test period. Such steps may be repeated at a desired frequency and duration for the remainder of the best period. A light dose, as measured by the radiant exposure measuring device, is logged.

### BRIEF DESCRIPTION OF THE DRAWINGS

Certain embodiments are shown in the drawings. However, it is understood that the present disclosure is not limited to the arrangements and instrumentality shown in the attached drawings.

FIG. 1 is perspective view of a prior art accelerated weathering test apparatus.

FIG. 2 is a detailed partially cut-away view of a portion of the apparatus of FIG. 1.

FIGS. 3A-C schematically represent one embodiment of a prior art sequence of operating the apparatus of FIG. 1.

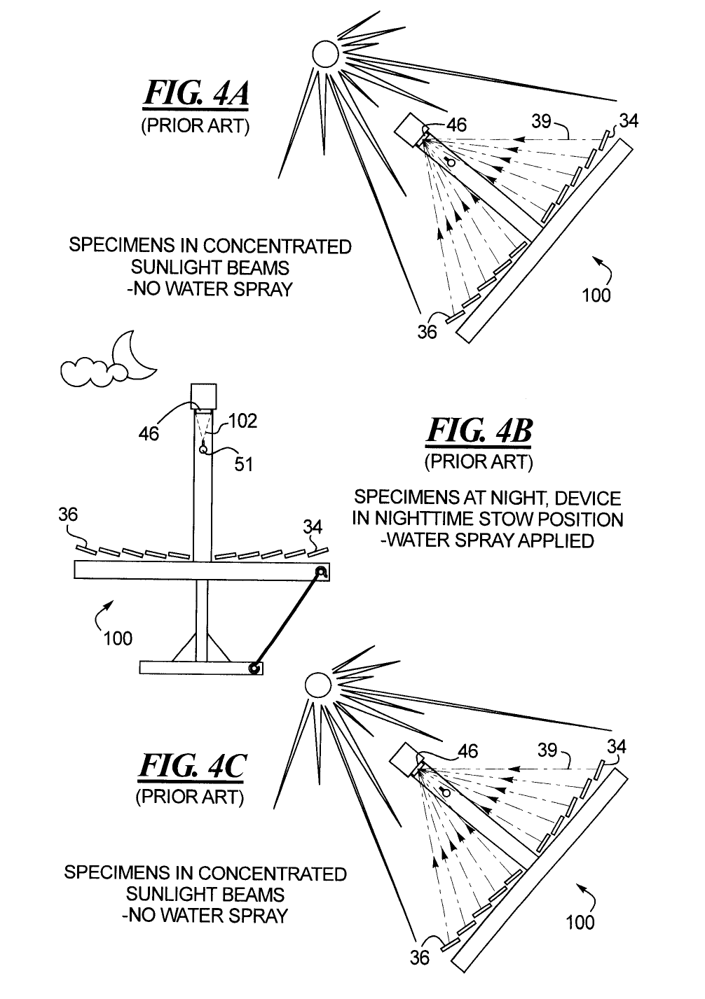

FIGS. 4A-C schematically represent another embodiment of a prior art sequence of operating the apparatus of FIG. 1.

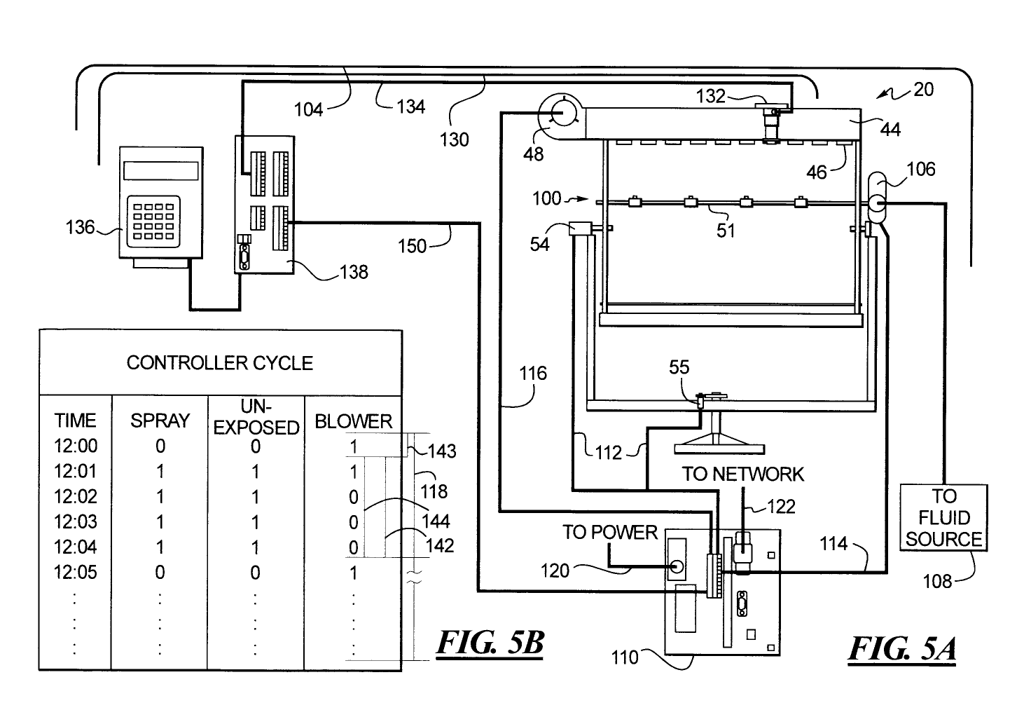

FIGS. 5A and 5B illustrate an accelerated weathering assembly in accordance with one embodiment of the present disclosure.

FIGS. 6A-C schematically represent a method of accelerated weathering in accordance with one embodiment of the present disclosure.

FIGS. 7A-C schematically represent a method of accelerated weathering in accordance with another embodiment of the present disclosure.

FIGS. 8A-C schematically represent a method of accelerated weathering in accordance with another embodiment of the present invention.

### DESCRIPTION OF THE PREFERRED EMBODIMENTS

For the purposes of promoting and understanding the principles disclosed herein, reference will now be made to the preferred embodiments illustrated in the drawings and specific language will be used to describe the same. It will nevertheless be understood that no limitation of the scope is thereby intended. Such alterations and further modifications in the illustrated devices and such further applications of the principles disclosed as illustrated herein as being contemplated as would normally occur to one skilled in the art to which this disclosure relates.

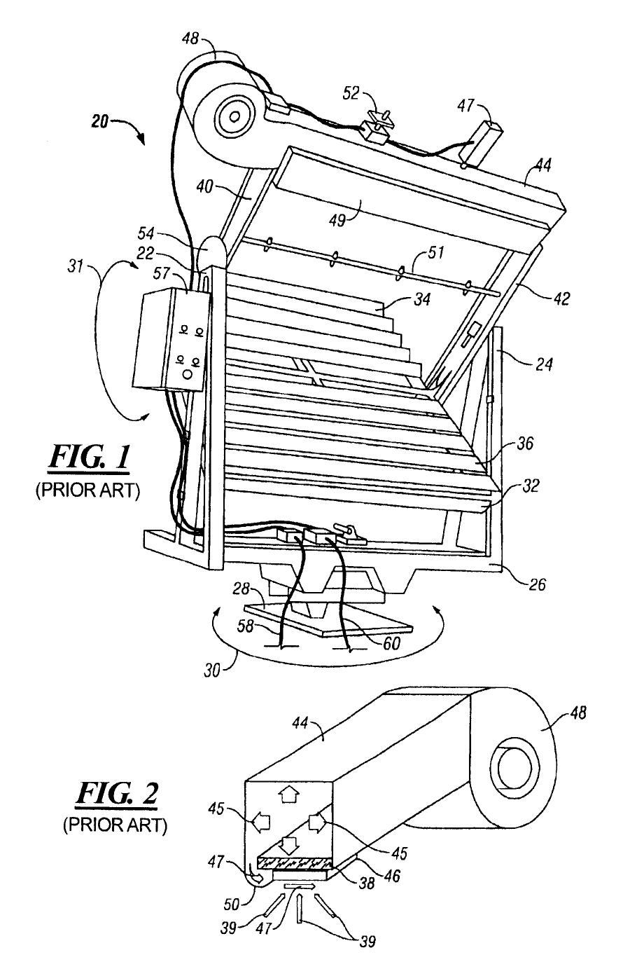

Referring to FIG. 1, a prior art accelerated weathering test apparatus is designated generally by reference 20 and includes a pair of A-frame members 22 and 24 to support the operative portion of the apparatus. The lower ends of the A-frame members 22, 24 are interconnected by a base member 26 which is operatively connected to a ground engaging member 28 in order to provide azimuth rotation in the direction indicated by arrow 30 and elevation rotation in the direction indicated by arrow 31. The elevation direction rotation accounts for periodic variation in the sun's altitude at solar noon. The azimuth direction rotation accounts for the east-west movements of the sun during the day.

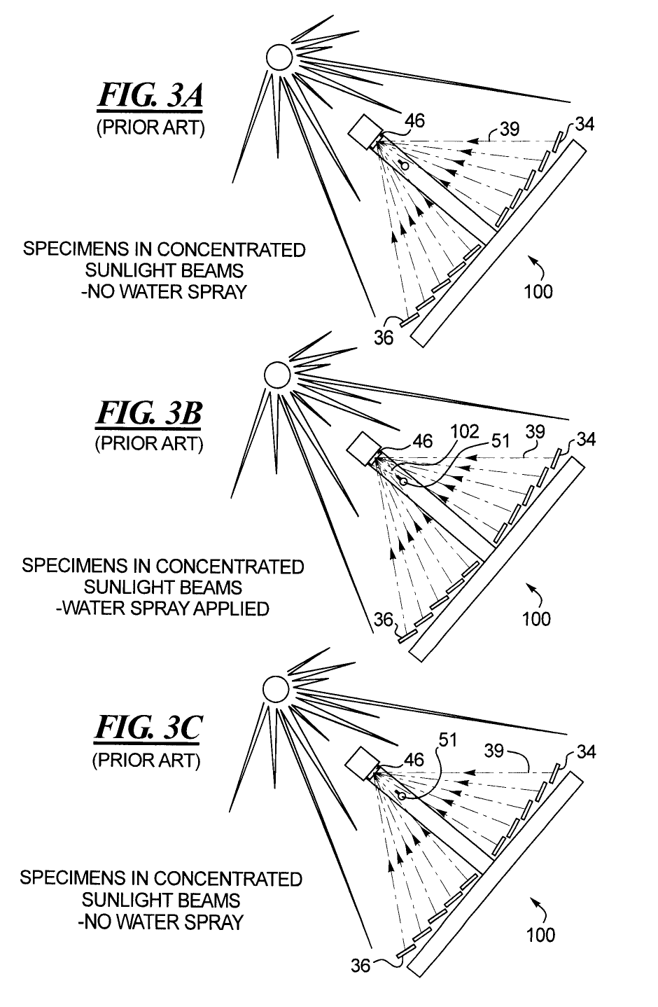

Rotatively supported from the upper ends of A-frame members 22, 24 is the operative portion of the apparatus preferably including a mirror bed frame 32 which supports a plurality of flat mirrors, including those designated by reference numerals 34 and 36. The plurality of mirrors 34, 36 are angled to reflect solar radiation directly impinging upon such mirrors to a target board 38 (see FIG. 2).

A pair of standards 40 and 42 extend outwardly from and perpendicular to mirror bed frame 32. An air tunnel 44 having a generally rectangular cross section is supported by the upper ends of standards 40, 42. Referring to FIG. 2, target board 38 is supported by the lower wall of air tunnel 44, and a plurality of test specimens 46 are mounted to the target board 38 for exposure to the concentrated solar radiation, represented in FIG. 2 by the upwardly extending arrows numbered 39. A squirrel cage blower assembly 48 communicates with one end of the air tunnel 44. Squirrel cage blower assembly 48 includes a fan driven by an electric motor to circulate cooling ambient air through air tunnel 44, represented in FIG. 2 by the outwardly extending arrows numbered 45. As shown in FIG. 2, air tunnel 44 includes a deflector 50 which extends for the length of target board 38 and causes cooling ambient air to be circulated across target board 38 for cooling test specimens 46, represented in FIG. 2 by the arrows numbered 47.

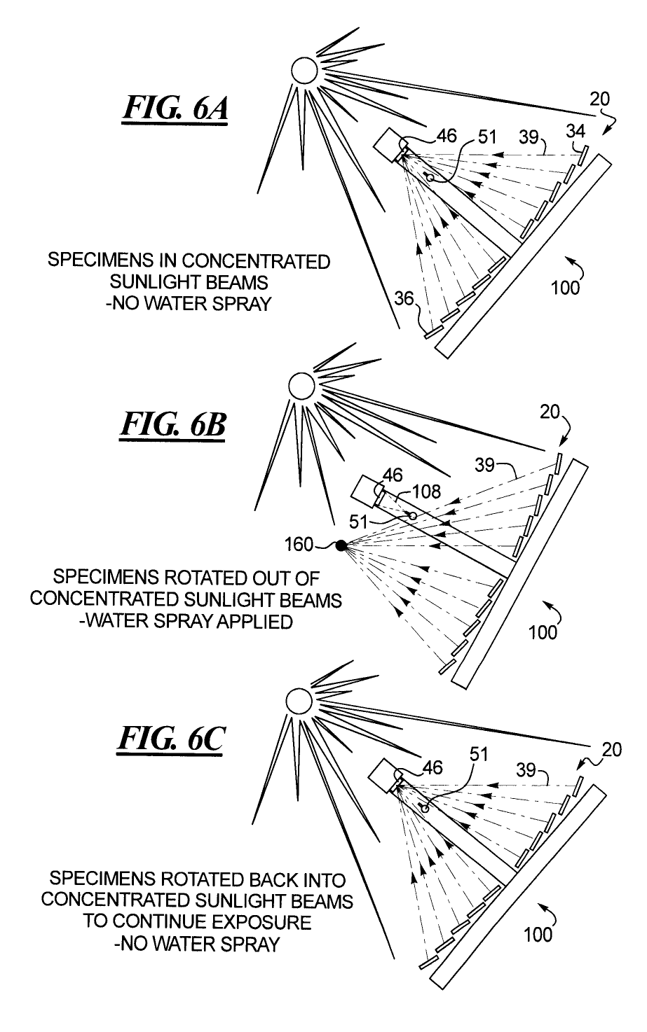

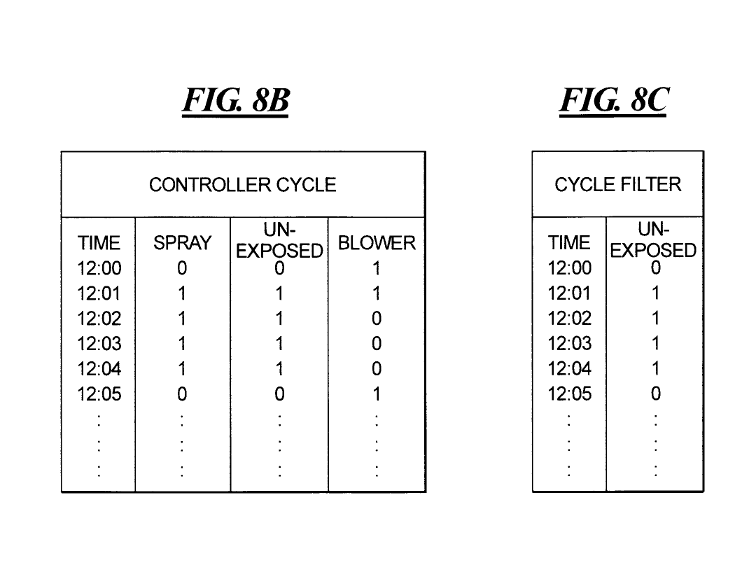

Standards 40, 42 are rotatively supported to upper ends of A-frame members 22, 24. A supporting shaft coincident with the axis of rotation in passing through standards 40, 42 rotably supports that portion of the test apparatus which tracks daily movements of the sun. In order to properly position the Fresnel-reflecting solar concentrator comprised by mirror assembly 34, 36 a reversible electric tracking servo-motor and related gear drive, generally designated by reference number 54 for altitude servo and 55 for the azimuth servo (not shown here, but in FIGS. 5A and 8A, disposed in the ground engaging member 28), are provided for periodically rotating the mirror bed and target board assembly to track movements of the sun. The clutch preferably couples standard 40 to a shaft to rotate the mirror assembly 34, 36 and target board assembly while permitting manual positioning of the unit at any time to reposition for maintenance. A solar cell tracking unit 52 controls the application of electrical power to the tracking servo-motor 54 or 55 in order to maintain the mirror bed frame 32 perpendicular to incident rays of sunlight. A solar tracker may be of the type which includes two balanced photo cells and a shadowing device mounted above such photo cells for shading them. When an imbalance is detected resulting from one photo cell receiving more sunlight than the other photo cell, an electrical error signal is generated which is amplified and used to apply power to the servo motor 54 or 55 for rotating the unit until the photo cells are again balanced, indicating that the unit is properly positioned with respect to the sun.

Also shown in FIG. 1 is a water spray nozzle assembly, designated generally by reference numeral 51. As shown in FIG. 1, spray nozzle assembly 51 is used to periodically spray water at the test specimens to simulate dew, rain, etc.

A hinge shield or cover 49 is shown coupled to the air tunnel 44 opposite the air deflector 50. A door release mechanism 47 is disposed on the air tunnel 44 for engaging and maintaining the shield in a closed position. Upon release, the shield 49 assumes the position shown in FIG. 1 such that concentrated solar radiation reflected by the plurality of mirrors 34, 36 reaches the test specimens 46.

Referring now to FIG. 2, the target board 38 is shown, including at least one test specimen 46 secured thereto. Only one test specimen is shown; however, a plurality are commonly used.

Referring again to FIG. 1, a controller box 57 houses the power and controller systems for the apparatus 20. A power cable 58 supplies electrical power to the apparatus 20 for powering the electric motor, which actuates the fan 48. A signal cable 60 is connected to the controller system disposed within the control box 57 for communication with remotely disposed devices, such as the feedback devices or input device, or for communication with a central command for governing the operation of the apparatus 20 in accordance with the present invention.

Referring to FIGS. 5A and 5B, an outdoor accelerated weathering assembly in accordance with one embodiment of the present disclosure is generally identified by a reference 104. An outdoor accelerated weathering test apparatus 20 is configured substantially as described above with respect to FIGS. 1 and 2 except for the differences described herein with respect to the improvements of the present disclosure. The outdoor accelerated weathering test apparatus 20 preferably comprises an operative portion 100 including a test specimen 46, a tracking servo-motors 54 and 55, a spray bar 51 and valve 106. The outdoor accelerated weathering test apparatus 20 may also further include a blower 48 and an air tunnel 44 for the purposes described above. As described above, the tracking servo-motors 54 and 55 are operatively connected to the operative portion 100 of the accelerated weathering test apparatus 20 in order to align the mirror assembly (not shown in detail in FIG. 5A, but identical to the mirrors shown and described with respect to FIG. 1) with the sun such that concentrated sunlight impinges upon the test specimens 46. Preferably, the water spray nozzle assembly including the spray bar 51 is disposed in opposition to the test specimens 46 and is selectively couple-able to a fluid source 108 by the valve 106.

A controller 110 is operatively coupled to the tracking servo-motors 54 and 55 by link 112 which provides signals to cause the tracking servo-motors 54 and 55 to adjust the position of the operative portion 100 with respect to the sun. It is within the teachings of the present disclosure that link 112 or any other link herein may be referred to as any suitable connection such as a hard-wired connection, signal cable, power and signal cable, wireless connection or the like depending on the desired application. It will be recognized by those of skill in the art that a separate power cable (not shown) may be provided for the tracking servo-motors 54 and 55 such that link 112 may be solely a control signal link, if so desired. In another embodiment, the controller 110 may also be operatively coupled to the valve 106 by link 114 and the blower 48 by link 116. Again, links 114 and 116 may be configured as desired and described above to fit any suitable installation configuration. The controller 110 is also connected to power via link 120 and may also be connected to a network via link 122. In another embodiment, the controller 110 may also be operatively connected to a recording device 138 by link 150.

Preferably, the controller 110 may be configured as a programmable logic controller comprising a processor and memory that stores programming instructions that when read by the processor causes the controller to function during a pre-selected test period 118. Other suitable controllers may be used, for example, a processing module including a processor and memory to facilitate management of the operations of the processing module. The processor may be a microprocessor, central processing unit or micro-controller, application-specific integrated circuit, field programmable gate array, a digital signal processor, a micro-controller or any other suitable processing device. If the processor is a microprocessor, it can be a “PENTIUM,” “POWER PC,” or any other suitable microprocessor, CPU or micro-controller commonly known in the art. The memory may be read-only memory, random access memory, rewritable disc memory, write-once-read-many disc memory, electrically erasable programmable ROM (EEPROM), holographic memory, remote storage memory or any other suitable memory device commonly known in the art. The memory includes instructions that are executed by the processor as well as programming variables or any other suitable programming source code or object code commonly known in the art.

A radiant exposure logging assembly 130 is preferably comprised of a radiant exposure measuring device 132 that is operatively coupled by cable 134 to a recording device 138. The recording device 138 logs a light dose that impinges on the test specimen 46 over the pre-selected test period 118 as measured by the radiant exposure measuring device 132. Preferably, the radiant exposure measuring device 132 may be a total ultraviolet radiometer, such as may be available from The Eppley Laboratory, Inc. in Newport, R.I. under model number TUVR, or any other suitable device or mechanism, such as a total solar radiometer, spectroradiometer or other like device. The recording device may be a controller as similarly described above or any suitable apparatus or device, that has a memory, read-only memory, random access memory, rewritable disc memory, write-once-read-many disc memory, electrically erasable programmable ROM (EEPROM), holographic memory, remote storage memory or any other suitable memory device commonly known in the art. The radiant exposure logging assembly 130 may further comprise a hardware or software filter (a functional representation is set forth in FIG. 8C) that causes the recording device 138 to cease measurement of the light dose measured by the radiant exposure measuring device 132 during an unexposed portion of the test period. As a software configuration, the filter may be a set of instructions stored in the memory of the recording device 138 for execution by the processor therein or any other suitable execution of software program stored in the recording device 138 as would be known to one of skill in the art. As a hardware configuration, the filter may be stored in the controller 110 as a series of instructions stored in the memory thereof for execution by the processor and as a result of link 150 the recording device 138 may be cycled on and off accordingly or any other suitable hardware device to provide or complete the identified function, such as a solenoid, switch, or the like.

In this embodiment, the radiant exposure measuring device 132 is disposed adjacent the test specimen 46 and exposed to concentrated sunlight. In another embodiment as shown in FIG. 8A, the radiant exposure measuring device 132 is adjacent the test specimen 46, but exposed to direct sunlight.

Referring to FIG. 5B, a controller cycle represents the programming instructions stored in the memory of the controller 110 that when read by the processor cause the controller 110 to function during the pre-selected test period 118. It is within the teachings of the present disclosure that the test period 118 may be configured in any suitable manner to perform a desired test. It will be recognized by those of skill in the art that the controller cycle represented herein is not limiting in any manner and that any other suitable cycle may be used. As shown in FIG. 5B, it will be recognized that with respect to the “Spray” column portion, the number 0 represents that the valve 106 has not been activated by the controller 110 and that fluid from the fluid source 108 is not being dispensed by the spray bar assembly 51, whereas the number “1” represents the opposite condition, i.e., controller 110 has activated the valve 106 so that fluid from the fluid source 108 is being dispensed from the spray bar assembly 51. With respect to the “unexposed” column portion, the number 0 represents that the controller 110 has activated the tracking servo-motors 54 and/or 55 so that the test specimen 46 is exposed to concentrated sunlight and the number “1” represents that the controller 110 has activated the tracking servo-motors 54 and/or 55 so that the test specimen 46 is removed from exposure to the concentrated sunlight. With respect to the “Blower” column portion, similarly the number “1” represents that the blower 48 has been activated by the controller 110, whereas the number “0” represents that the controller 110 has deactivated the blower 48.

The programming instructions stored in the memory, that, when read by the processor, cause the controller to function during the pre-selected test period 118 to actuate the tracking servo-motors 54 and/or 55 so that the test specimen 46 is exposed to concentrated sunlight for a pre-selected exposure portion 140 of the pre-selected test period 118. The programming instructions further cause the controller to function to actuate the tracking servo-motors 54 and/or 55 so that the test specimen is removed from exposure to the concentrated sunlight for a pre-selected unexposed portion 142 of the pre-selected test period 118. Each of the above steps may be repeated for the remainder of the pre-selected test period 118. It will be recognized that integration of operation of the recording device 138 into the operation of the controller is of key importance to this disclosure so that an accurate account of the light dose or radiant exposure impinging on the test specimen 46 is recorded.

In this embodiment, the link 150 between the controller 110 and the recording device 138 is not necessary because the radiant exposure measuring device 132 is disposed as would be a test specimen 46. Accordingly, when the test specimen 46 is exposed to sunlight or concentrated sunlight the output of the radiant exposure measuring device 132 logged by the recording device 138 will be represented by a large signal. However, when the test specimen 46 is moved out of exposure to concentrated sunlight or sunlight the output of the radiant exposure measuring device 132 logged by the recording device 138 will be represented by a small signal. However, it will be recognized that the link 150 may be useful to control operation of the recording device 138.

As further shown in FIG. 5B, the controller 110 may include further programming instructions, that, when read by the processor, cause the controller to function to actuate the valve 106 such that fluid from the fluid source 108 is dispensed by the spray bar 51 onto the test specimen during a portion 144 of the pre-selected unexposed portion 142 of the pre-selected test period 118. Moreover, the controller may include further programming instructions, that, when read by the processor, cause the controller to function to actuate the blower 48. The blower may be variably controlled, for example, in accordance with the teachings of U.S. Pat. No. 6,659,638, incorporated herein by reference. Additionally, the blower 48 may be actuated as shown in the pre-selected exposure period 140. In fact, as shown in pre-selected test period 118, the blower 48 may be actuated before the pre-selected unexposed portion 142 of the pre-selected test period 118 and before the pre-selected exposure portion of the pre-selected test period 118. It is within the teachings of the present disclosure that reference to actuation of the blower may be on, off, any variable setting therebetween, or any other suitable functionality.

Referring to FIGS. 8A-C, another embodiment of an outdoor accelerated weathering assembly in accordance with the principal aspects of the present disclosure is shown. This embodiment is substantially identical to the embodiment described in FIGS. 5A and 5B except as noted herein. Similarities will not be repeated for the sake of brevity, however such similarities are incorporated into this embodiment by reference.

The principal differences in this embodiment is that the radiant exposure measuring device 132 that is disposed adjacent to test specimen 46 is exposed to direct sunlight as opposed to concentrated sunlight and that the recording device 138 is connected to the controller 110 by link 150. Moreover, as shown in FIG. 8C, the operation of the filter 138 is illustrated. One of skill in the art will note that the filter cycle corresponds to the controller cycle as shown in FIG. 8B, on the basis of time, such that the operation of the filter cycle and the controller cycle correspond time-wise. Synchronization on the basis of time may be provided by individual timers in the controller and filter or each may be liked to a network (LAN, WAN, etc.) which has a single, centralized timekeeper in any suitable manner (i.e., hard wired, wireless or the like). It is within the teachings of the present invention that the filter ceases measurement of the light dose in accordance with the teachings herein whenever the controller has actuated the tracking servo-motors 54 and/or 55 so that the test specimen 46 is removed from exposure to the concentrated sunlight, as indicated by the number “1”. As a result, the recording device 138 logs only the light dose to which the test specimen 46 is exposed directly rather than the total light dose of which the radiant exposure measuring device 132 is exposed to. Interface device 136 may be used with any of the embodiments of this disclosure to facilitate operation of the recording device 138 and change or alter any operation parameters thereof as may be desired.

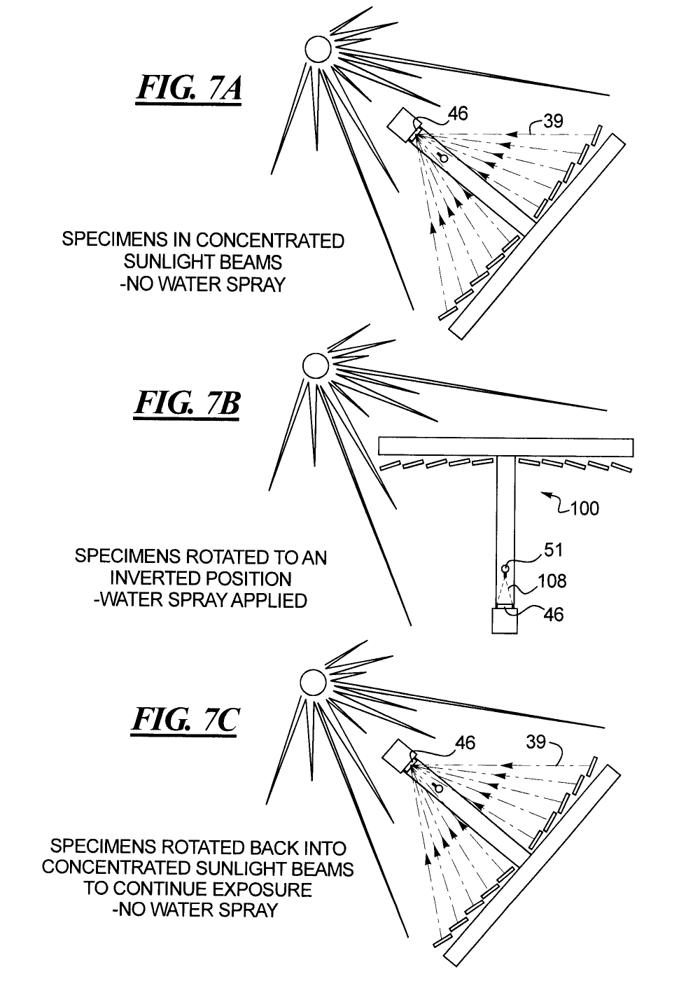

Referring to FIGS. 6A-C, one embodiment of a method for outdoor accelerated weathering of a test specimen 46 disposed on an accelerated weathering test apparatus 20 for the pre-selected secondary test period is set forth and may be executed in connection with either embodiment described above with respect to FIGS. 5A-C and 8A-C. In this embodiment, the accelerated weathering test apparatus 20 comprises an operative portion 100 and tracking servo-motors (not shown) operatively coupled to the operative portion 100. A radiant exposure measuring device is disposed adjacent the test specimen 46, either exposed to concentrated sunlight or direct sunlight, as may be desired, for the test period. A recording device is also linked to the radiant exposure measuring device and integrated into operation of the controller.

In FIG. 6A, the programming instructions stored in the memory of the controller are read by the processor and cause the controller to function to actuate the tracking servo-motor so that the test specimen 46 is exposed to concentrated sunlight 39 for the pre-selected exposure portion of the pre-selected test period. One of skill in the art will note the focal point of the mirrors 34, 36 is on the test specimen 46. Moreover, the fluid spray from the spray bar assembly 51 has not been activated. The recording device also begins to record or log data when either the signal from the radiant exposure measuring device reaches a threshold level or after being activated or actuated by software or hardware resident in the recording device or the controller as the case may be.

In FIG. 6B, the tracking servo-motors have been actuated by the controller such that the test specimen 46 is removed from exposure to the concentrated sunlight for a pre-selected unexposed portion of the pre-selected test period. A dot 160 illustrates the focal point of the concentrated sunlight 39 which has been moved away from the test specimen as a result of movement of the operative portion 100 with respect to the sun. Moving the focal point of the concentrated sunlight 39 to dot 160 permits the test specimen 46 to cool naturally or with the aid of the blower, as may be desired. After a suitable cooling time, the programming instructions, that, when read by the processor, will cause the controller to function to actuate the valve such that fluid 108 from the fluid source is dispensed by the spray bar assembly 51 onto the test specimen 46 during a portion of the pre-selected unexposed portion of the pre-selected test period. The recording device stops recording or logging data when the signal from the radiant exposure measuring device drops below the threshold level or when deactivated or stopped by the hardware or software resident in the recording device or controller as the case may be. Thereby, overcoming all the disadvantages of the prior art described above.

In FIG. 6C, the steps described above have begun repeating, namely, that the tracking servo-motors have been actuated so that the test specimen 46 is exposed to concentrated sunlight 39 for another pre-selected exposure portion of the pre-selected test period. Additionally, the spray bar assembly 51 has been deactivated such that more fluid from the fluid source is not dispensed on the test specimen 46 when exposed to concentrated sunlight. The blower may also be activated during any period in which the test specimen 46 is exposed. It is also within the teachings of the present disclosure that the blower may be actuated, in any manner described herein, before the pre-selected unexposed portion of the pre-selected test period and/or before the pre-selected exposure portion of the pre-selected test period as may be desired or suitable for selected tests. Again, the recording device is activated or actuated as described above.

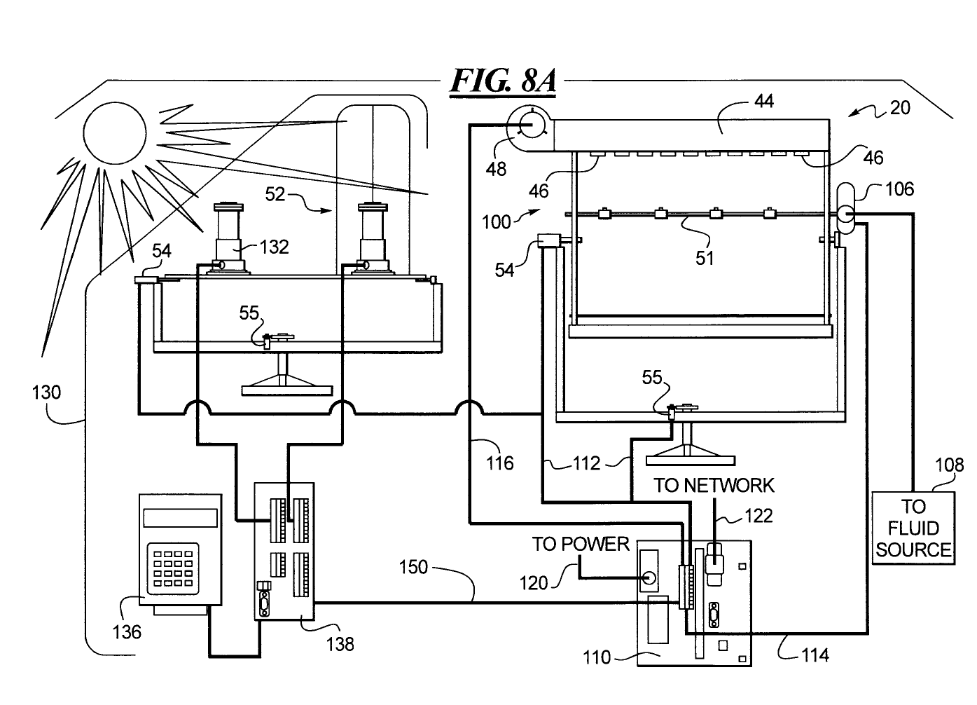

Referring to FIGS. 7A-C, another embodiment of a method for accelerated weathering of a test specimen in accordance with the principal aspects of the present disclosure is shown and is applicable with any of the embodiments disclosed herein. In FIG. 7A, as identically shown in FIG. 6A, the tracking servo-motor has been actuated by the controller so that the test specimen 46 is exposed to concentrated sunlight 39 for a pre-selected exposure portion of the pre-selected test period. Likewise, the recording device begins to record or log data when either the signal from the radiant exposure measuring device reaches a threshold level or after being activated or actuated by software or hardware resident in the recording device or the controller as the case may be.

In FIG. 7B, similar to the steps described in FIG. 6B, the tracking servo-motors are actuated by the controller so that the test specimen 46 is removed from exposure to the concentrated sunlight for a pre-selected unexposed portion of the pre-selected test period. However, the operative portion 100, in this embodiment, has been inverted such that the spray bar assembly 51 when actuated to dispense fluid 108 onto the test specimen 46 such fluid 108 is dispensed vertically downward. As a result, the test specimen 46 is disposed generally horizontal such that the fluid 108 will pool thereon in accordance with the desired test parameters. Again, the recording device stops recording or logging data when the signal from the radiant exposure measuring device drops below the threshold level or when deactivated or stopped by the hardware or software resident in the recording device or controller as the case may be.

In FIG. 7C, the controller has actuated the tracking servo-motors such that the test specimen 46 is exposed to concentrated sunlight 39 as described above with respect to FIG. 6C and the recording device resumes recording or logging data as described above.

While the particular preferred embodiments have been shown and described, it will be obvious to those skilled in the art that changes and modifications may be made without departing from the teaching of the disclosure. For example, materials of construction may be changed or altered to perform the functions disclosed herein without departing from the nature of this disclosure. Mechanical or optical control devices may be substituted for the control and input signals and that other methods to effect temperature using the mirrors rather than the blown air may be used. For instance, defocusing mirrors instead of changing the blowers speed may provide the same results. Additionally, a damper or mechanical valve in the air tunnel may be used to change the amount of cooling air circulated over the test specimen. Finally, filters (polarizing, interference, tunable, etc.) may be used to effect the radiance. The matter set forth in the foregoing description and accompanying drawings is offered by way of illustration only and not as limitation. The actual scope of the disclosure is intended to be defined in the following claims when viewed in their proper perspective based on the related art.

What is claimed is:

1. An outdoor accelerated weathering assembly comprising:
(a) an outdoor accelerated weathering test apparatus comprising an operative portion including:
(i) a test specimen; and
(ii) a tracking servo-motor operatively coupled to the operative portion;
(b) a controller operatively coupled to the tracking servo-motor, the controller comprising a processor and memory that stores programming instructions, that, when read by the processor, cause the controller to function during a pre-selected test period to:
(i) actuate the tracking servo-motor so that the test specimen is exposed to concentrated sunlight for a pre-selected exposure portion of the pre-selected test period;
(ii) actuate the tracking servo-motor so that the test specimen is removed from exposure to the concentrated sunlight for a pre-selected unexposed portion of the pre-selected test period; and
(iii) repeat steps (i) and (ii) for a remainder of the pre-selected test period; and
(c) a radiant exposure logging assembly comprising a radiant exposure measuring device operatively coupled to a recording device, wherein the recording device logs a light dose that impinges on the test specimen over the pre-selected test period as measured by the radiant exposure measuring device.

2. The outdoor accelerated weathering assembly as recited in claim 1, further comprising a spray bar disposed in opposition to the test specimen, the spray bar selectively couple-able to a fluid source by a valve.

3. The outdoor accelerated weathering assembly as recited in claim 2, wherein the controller is operatively coupled to the valve.

4. The outdoor accelerated weathering assembly as recited in claim 3, wherein further programming instructions, that, when read by the processor, cause the controller to function to actuate the valve such that fluid from the fluid source is dispensed by the spray bar onto the test specimen during a portion of the pre-selected unexposed portion of the pre-selected test period.

5. The outdoor accelerated weathering assembly as recited in claim 1, wherein the outdoor accelerated weathering test apparatus further comprises a blower operatively coupled to an air tunnel having an outlet adjacent the test specimen and the controller comprises further programming instructions, that, when read by the processor, cause the controller to function to actuate the blower.

6. The outdoor accelerated weathering assembly as recited in claim 5, wherein the blower is actuated before the pre-selected unexposed portion of the pre-selected test period.

7. The outdoor accelerated weathering assembly as recited in claim 5, wherein the blower is actuated before the pre-selected exposure portion of the pre-selected test period.

8. The outdoor accelerated weathering assembly as recited in claim 1, wherein the radiant exposure measuring device is exposed to concentrated sunlight.

9. The outdoor accelerated weathering assembly as recited in claim 1, wherein the radiant exposure measuring device is exposed to direct sunlight.

10. The outdoor accelerated weathering assembly as recited in claim 1, wherein the radiant exposure logging assembly further comprises a filter that causes the recording device to cease measurement the light dose during the pre-selected unexposed portion of the pre-selected test period.

11. The outdoor accelerated weathering assembly as recited in claim 4, wherein the test specimen is disposed generally horizontal such that the fluid pools thereon.

12. A method for accelerated weathering of a test specimen disposed on an accelerated weathering test apparatus for a pre-selected test period, the accelerated weathering test apparatus comprising an operative portion and a tracking servo-motor operatively coupled to the operative portion, the method comprising:
(a) providing a radiant exposure measuring device adjacent the test specimen;
(b) coupling a recording device to the radiant exposure measuring device;
(c) coupling a controller to the tracking servo-motor, the controller including a processor and memory that stores programming instructions, that, when read by the processor, cause the controller to function to;
(i) actuate the tracking servo-motor so that the test specimen is exposed to concentrated sunlight for a pre-selected exposure portion of the pre-selected test period;
(ii) actuate the tracking servo-motor so that the test specimen is removed from exposure to the concentrated sunlight for a pre-selected unexposed portion of the pre-selected test period; and
(iii) repeat steps (i) and (ii) for a remainder of the pre-selected test period; and
(d) logging a light dose measured by the radiant exposure measuring device that impinges on the test specimen over the pre-selected test period with the recording device.

13. The method as recited in claim 12, further comprising: providing a spray bar disposed in opposition to the test specimen.

14. The method as recited in claim 13, further comprising: coupling the spray bar to a fluid source by a valve.

15. The method as recited in claim 14, further comprising: coupling the controller to the valve.

16. The method as recited in claim 15, wherein the controller comprises further programming instructions, that, when, read by the processor, cause the controller to function to actuate the valve such that fluid from the fluid source is dispensed by the spray bar onto the test specimen during a portion of the pre-selected unexposed portion of the pre-selected test period.

17. The method as recited in claim 12, wherein the outdoor accelerated weathering test apparatus further comprises a blower operatively coupled to an air tunnel having an outlet adjacent the test specimen and the controller comprises further programming instructions, that, when read by the processor, cause the controller to function to actuate the blower.

18. The method as recited in claim 17, wherein the blower is actuated before the pre-selected unexposed portion of the pre-selected test period.

19. The method as recited in claim 17, wherein the blower is actuated before the pre-selected exposure portion of the pre-selected test period.

20. The method as recited in claim 12, wherein the radiant exposure measuring device is exposed to concentrated sunlight.

21. The method as recited in claim 12, wherein the radiant exposure measuring device is exposed to direct sunlight.

22. The method as recited in claim 21, wherein the radiant exposure logging assembly further comprises a filter that causes the recording device to cease measurement the light dose during the pre-selected unexposed portion of the pre-selected test period.

23. The method as recited in claim 16, wherein the test specimen is disposed generally horizontal such that the fluid pools thereon.
